(19) United States
(12) Patent Application Publication
(10) Pub. No.: US 2004/0121464 A1
(43) Pub. Date: Jun. 24, 2004

(54) Method for the preparation of cells of mesodermal lineage

(76) Inventors: Peter David Rathjen, Mitcham (AU); Joy Rathjen, Mitcham (AU); Nathan Tobias Harvey, Dernan court (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/675,938

(22) Filed: Sep. 30, 2003

Related U.S. Application Data
(60) Provisional application No. 60/414,959, filed on Sep. 30, 2002.

(51) Int. Cl. C12N 5/08; C12N 15/85

(52) U.S. Cl. 435/455; 435/366

(57) ABSTRACT

The present invention relates generally to the generation of cells of mesodermal lineage. More particularly, the present invention contemplates a method for the preparation of differentiated or partially differentiated mesodermal cells and their use in tissue repair, regeneration and/or augmentation therapy. The identification and generation of the mesodermal cells further provides a source of transcriptome or proteome data to assess the expression profile of genes associated with the maintenance of mesodermal cells as well as their differentiation, proliferation, expansion and/or renewal potential.

### RELATED APPLICATION

This application is a non-provisional application of Provisional Application No. 60/414,959, filed Sep. 30, 2002 the entire disclosure of which is expressly incorporated herein by reference in its entirety.

### BACKGROUND OF THE INVENTION

1. Field of the Invention

The present invention relates generally to the generation of cells of mesodermal lineage. More particularly, the present invention contemplates a method for the preparation of differentiated or partially differentiated mesodermal cells and their use in tissue repair, regeneration and/or augmentation therapy. The identification and generation of the mesodermal cells further provides a source of transcriptome or proteome data to assess the expression profile of genes associated with the maintenance of mesodermal cells as well as their differentiation, proliferation, expansion and/or renewal potential.

2. Description of the Related Art

Bibliographic details of references provided in the subject specification are listed at the end of the specification.

Reference to any prior art in this specification is not, and should not be taken as, an acknowledgment or any form of suggestion that this prior art forms part of the common general knowledge in any country.

Initial developmental events within the mammalian embryo entail the elaboration of extra-embryonic cell lineages and result in the formation of the blastocyst, which comprises trophectoderm, primitive endoderm and a pool of pluripotent cells referred to as the inner cell mass (ICM/epiblast). As development continues, the cells of the ICM/epiblast undergo rapid proliferation, selective apoptosis, differentiation and reorganization as they develop to form the primitive ectoderm. In the mouse, the cells of the ICM begin to proliferate rapidly around the time of blastocyst implantation. The resulting pluripotent cell mass expands into the blastocoele or blastocoelic cavity. Between 5.0 and 5.5 days post coitum (dpc), the ICM of the epiblast undergoes apoptosis to form the proamniotic cavity. The outer, surviving cells, or early primitive ectoderm, continue to proliferate and by 6.0-6.5 dpc have formed a pseudo-stratified epithelial layer of pluripotent cells, termed the primitive or embryonic ectoderm. Primitive endoderm cells are pluripotent, distinct from cells of the ICM, and give rise to the germ cells. They also act as a substrate for the generation of the primary germ layers of the embryo proper (mesoderm, endoderm and ectoderm) and the extra-embryonic mesoderm during gastrulation.

By 4.5 dpc, pluripotent cells exposed to the blastocoele or blastocoelic cavity have differentiated to form primitive endoderm. The primitive endoderm gives rise to two distinct endodermal cell populations, visceral endoderm, which remains in contact with the epiblast, and parietal endoderm, which migrates away from the pluripotent cells to form a layer of endoderm adjacent to the trophectoderm.

Formation of these endodermal layers is coincident with the formation of primitive ectoderm and the creation of an inner cavity.

In the human and in other mammals, formation of the blastocyst, including development of ICM cells and their progression to pluripotent cells of the primitive ectoderm and subsequent differentiation to form the embryonic germ layers, follow a similar development process.

Pluripotent cells can be isolated from the preimplantation mouse embryo as embryonic stem (ES) cells. ES cells can be maintained indefinitely as a pluripotent cell population in vitro, and, when reintroduced into a host blastocyst, can contribute to all adult tissues of the mouse including the germ cells. ES cells, therefore, retain the ability to respond to all the signals that regulate normal mouse development and potentially represent a powerful model system for the investigation of mechanisms underlying pluripotent cell biology and differentiation within the early embryo, as well as providing opportunities for embryo manipulation with resultant commercial, medical and agricultural applications. ES cells and other pluripotent cells and cell lines will share some or all of these properties and applications.

The differentiation of ES cells can be regulated in vitro by various agents such as by the cytokine, leukemia inhibitory factor (LIF), and other gp130 agonists which promote self-renewal and prevent differentiation of the stem cells. However, there is little information about biological molecules that can induce the differentiation of ES cells into specific cell types.

Differentiation of ES cells to primitive ectoderm-like cells can be achieved by aggregation and culture for 4 days in a conditioned medium MEDII (see International patent applications PCT/AU99/00265 & WO01/51611). Continued culture in MEDII, followed by culture in defined serum free medium results in formation of a population of cellular aggregates comprised entirely of neurectoderm. This has been demonstrated morphologically with the formation of neurons (ectoderm) but not beating cardiocytes (mesoderm), and by gene expression analysis, with the expression of Sox1, Sox2, nestin and N-Cam, early neural specific markers but not brachyury, an early mesodermal marker or markers of the extraembyonic endodermal lineage SPARC or alpha-feto protein. Furthermore, applicants have shown that the neural progenitor cells formed during EPL cells differentiation can be directed to form alternate neural cell lineages, such as neural crest and glia, by the addition of exogenous signalling molecules.

There is a need, therefore, to develop protocols for the control and/or modulation of the differentiation process in relation to EPL cells as well as other uncommitted cells or groups of cells from pre- or post-natal animals. In particular, the ability to generate mesodermal cells from EPL cells or other stem cells would greatly facilitate the repair, regeneration and/or augmentation of the haemopoietic lineages, muscle lineages, bone and connective tissue and organ tissue such as liver, pancreas and kidney tissue as well as brain, epidermis skin, breast, lung, muscle, heart, eye, bone, spleen, gut, biliary system, various portions of the evaginated structures, thyroid gland, thymus and epithelium and cells of the immune system.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated in part on the elucidation of the signalling required to generate mesodermal cells from early primitive ectoderm-like (EPL) cells and also potentially from other non-committed cells. In particular, the present invention identifies bone morphogenetic protein 4 (BMP4) as a critical component in the signalling process in the differentiation of inter alia EPL cells into mesodermal cells.

The present invention contemplates, therefore, a method for generating or otherwise enriching a population of mesodermal cells from a population of stem cells. The method comprises culturing stem cells or progenitor cells with an effective amount of BMP4 or a homologue, analogue or functional equivalent thereof. The BMP4 is required in an amount effective to induce differentiation of stem cells into mesodermal cells. Although the present invention is described herein with respect to EPL cells, the scope of the subject invention extends to other non-committed cells including ES cells and adult stem cells.

The culturing process is preferably conducted in Gibco Dulbecco's Modified Eagle Medium (DMEM) supplemented with BMP4. However, the present invention extends to any suitable medium which, in the presence of BMP4, does not induce EPL cell differentiation into ectodermal or endodermal cells.

Reference herein to “EPL cells” or “mesodermal cells” includes reference to EPL-like cells or mesodermal-like cells or cells which are committed to differentiate into EPL cells or mesodermal cells. Other non-committed cells are also contemplated such as ES cells.

In a preferred embodiment, the present invention provides a method for generating mesodermal cells from ES or EPL cells said method comprising:

(a) culturing ES cells or EPL cells in MEDII or its functional equivalent in order to generate embryoid bodies (EBM);

(b) maintaining the EBMs in culture for a time sufficient to allow aggregation of said EBMs;

(c) transferring the aggregated EBMs to gelatin-treated wells;

(d) allowing the aggregated EBMs to adhere to the gelatin-treated wells; and

(e) culturing the adhered EBMs in serum free medium comprising BMP4 for a time sufficient to allow the EBMs to generate mesodermal cells, and thereby generating mesodermal cells from ES cells or EPL cells.

The present invention further provides an isolated mesodermal cell or group of cells or a substantially homogenous culture or a substantially enriched population of mesodermal cells or their committed progenitor cells generated by culturing stem cells in the presence of BMP4.

The present invention further extends to the generation of mesodermal tissue from other non-committed cells, such as ES cells.

The ability to preferentially control differentiation of EPL cells into mesodermal cells enables the development of tissue repair, regeneration and/or augmentation therapies of haemopoietic lineage, muscle lineage, bone and connective tissue and organ tissue such as liver, pancreas and kidney tissue as well as brain, epidermus skin, breast, lung, muscle, heart, eye, bone, spleen, gut, biliary system, various portions of the evaginated structures, thyroid gland, thymus and epithelium and cells of the immune system.

Furthermore, the present invention leads to alternative therapies for disease conditions such as heart disease, blood diseases such as thalassemias or immune deficiencies or a range of other conditions. The therapeutic protocols include generating tissue in vitro or ex vivo for transplantation into the same or a different host from where the cells are isolated certain in vivo therapeutic protocols may also be employed.

The present invention further enables screening for agents which have functional properties analogous or similar to BMP4 in terms of promoting stem cell differentiation into mesodermal cells. Such agents may be identified following transcriptome or proteome analysis or following natural product screening or the screening of chemical libraries. These agents as well as BMP4 may be used to generate tissue in vitro, ex vivo or in vivo for tissue repair, regeneration and/or augmentation therapy.

Furthermore, the identification of the mesodermal lineage cells permits transcriptome or proteome determination to assess which genes are essential for the maintenance of an undifferentiated state or a partially differentiated state of mesodermal cells as well as which genes are required for differentiation, proliferation, expansion and/or renewal of stem cells or mesodermal cells. The identification of such genes then provides validated drug targets.

A list of abbreviations used in the subject specification together with definitions is provided in Table 1.

TABLE 1
Abbreviations

| Abbreviation | Definition |
|---|---|
| APS | ammonium persulphate |
| BSA | bovine serum albumin |
| BMP4 | Bone morphogenetic protein 4 |
| d.p.c. | Days post coitum |
| DMEM | Gibco Dulbecco's Modified Eagle Medium |
| EDTA | ethylenediaminetetra-acetic acid |
| ES cells | Embryonic stem cells |
| EPL cells | Early primitive ectoderm-like cells |
| EB | Embryoid bodies |
| EMB | EB formed in MEDII |
| EMB⁴ | EMB cultured for 4 days |
| EPLEB | EPL cell-derived embryoid bodies |
| FCS | foetal calf serum |
| HEPES | N-2-hydroxyethyl piperazine-N-ethane sulphonic acid |
| HRP | horse radish peroxidase |
| IP | immunoprecipitation |
| kD | kilodalton |
| LIF | leukemia inhibitory factor |
| mA | milliamperes |
| MEDII | Conditioned medium from HepG2 cells |
| PAGE | polyacrylamide gel electrophoresis |
| PBS | phosphate buffered saline |
| PBT | phosphate buffered saline + 0.1% Tween-20 |
| rpm | revolutions per minute |
| SDS | sodium dodecyl phosphate |
| sf MEDII-cFN | MEDII medium without cFN |
| TBST | tris buffered saline + Tween-20 |
| TEMED | N,N,N′,N′-teramethyl-ethenediamine |
| Tween-20 | polyoxyethylenesorbitan monolaurate |
| v | volume |
| w | weight |

### BRIEF DESCRIPTION OF THE DRAWINGS

FIG. 1 is a graphic representation showing (A) neuron and (B) cardiomyocyte formation. EBM⁴ were seeded as individual aggregates and grown with or without 10 ng/ml BMP4 until day 10 to 12. At this stage they were scored for the presence of neurons or beating cardiomyocytes. (A) Average percentage of aggregates showing neuron formation when grown either with or without BMP4 (n=7, error bars=standard error). (B) Average percentage of aggregates showing cardiomyocyte formation when grown either with or without BMP4 (n=7, error bars=standard error).

FIG. 2 is a photographic representation showing gene expression analysis of EBM⁴, seeded and differentiated either with 10 ng/mL BMP4 (C, D, E, F) or without BMP4 (A, B, G, H). Wholemount in situ hybridization analysis has been performed with digoxigenin-labelled antisense probes to brachyury (A, C, D) and Oct4 (B, E, F). Photos are taken using phase contrast microscopy. Sense probes were negative for both brachyury (G) and Oct4 (H).

FIG. 3 is a photographic representation showing phosphylation analysis of cell lysates isolated from EBM cultures in the presence or absence of BMP4. (A) Cell lysates from EBM⁴s that had been grown with or without 10 ng/mL of BMP4 for 15, 30, 45 and 120 minutes were subjected to SDS-PAGE and Western Blotting using an anti-phosphoSmad primary antibody (Cell Signaling Technology). Cell lysate from seeded EBM4s before addition of serum free media is also included (time=0). (B) The same membrane as in A re-probed with an anti-Actin primary antibody to confirm that similar amounts of lysate were added to each well. Expected size of Smad 1 and Smad 5 is 60 kD, of Smad 8 is 48 kD.

FIG. 4 is a photographic representation of SDS-PAGE and Western Blot of immunoprecipitates of cell lysates from EBM⁴s that had been grown with 10 ng/mL of BMP4 for 30 minutes. Lysates had been subjected to immunoprecipitation with an anti-phosphoSmad antibody, an irrelevant antibody (anti-Ecadherin, Santa Cruz) or no antibody. Membranes have been probed with antibodies against (A) Smad 1 (B) Smad 5 (C) Smad 8. Cell lysates from EBM6 have been included as positive controls—the position of the relevant Smad protein is indicated by the arrow. The same membranes were re-probed with anti-phopshoSmad antibody to confirm that similar amounts of immunoprecipitates had been loaded (D-E, relating to A-C respectively).

### DETAILED DESCRIPTION OF THE PREFERRED EMBODIMENT

The present invention is predicated in part on elucidation of a protein-mediated signalling process during gastrulation of the mammalian embryo. Gastrulation is the process whereby a population of uncommitted pluripotent progenitor cells differentiate to one of the three primary germ layers, ectoderm, endoderm or mesoderm. In accordance with the present invention, EPL cells are preferentially directed along the mesodermal lineage when cultured or otherwise exposed to BMP4. The present invention further extends to directing other stem cells such as ES cells or adult stem cells along a mesodermal lineage.

Accordingly, one aspect of the present invention contemplates a method for directing a population of stem cells to differentiate along an mesodermal cell lineage, said method comprising, culturing said stem cells in the presence of BMP4 or a homologue, analogue or functional equivalent thereof for a time and under conditions sufficient for stem cells to preferentially differentiate into mesodermal cells or cells of an mesodermal lineage.

Another aspect of the present invention provides a method for directing a population of EPL cells to differentiate along a mesodermal cell lineage, said method comprising, culturing said EPL cells in the presence of BMP4 or a homologue, analogue or functional equivalent thereof for a time and under conditions sufficient for EPL cells to preferentially differentiate into mesodermal cells or cells of a mesodermal lineage.

Reference to “BMP4” includes recombinant, synthetic or purified, naturally occurring BMP4 as well as homologues, analogues or chemical or functional equivalents thereof. The preparation, however, does not comprise signalling molecules which cause differentiation of EPL cells or other stem cells into ectodermal or endodermal cells.

BMP4 is generally derived from the same species of mammal from which the EPL cells are isolated. In this case, the BMP4 is said to be homologous to the stem cells. However, the present invention extends to the use of heterologous BMP4 where the BMP4 is derived from a mammalian species different from mammalian species from which the stem cells are isolated. Mammalianized such as humanized BMP4 is also contemplated by the present invention. For example, where human stem cells are desired to be directed to mesodermal cells, humanized porcine BMP4 or humanized ovine BMP4 may be used. In another example, the BMP4 is derived from a human cell line and the experimental model is mouse ES cells.

A chemical analogue of BMP4 is also contemplated.

All chemical modifications to BMP4 molecules or other functionally equivalent growth factors as well as the generation of parts, fragments, portions, derivatives or homologs thereof, are contemplated by the present invention. A BMP4 may, for example, be considered pleiotropic and have multiple and sometimes conflicting activities. A fragment or derivative of a BMP4 may exhibit a particularly desired activity while losing a non-desired activity. Reference herein to a “BMP4” includes analogs and in particular chemical analogs including chemical modifications to side chains.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, omithine, sarcosine, 4-amino-3-hydroxy-6-methylheptanoic acid, 2-thienyl alanine and/or D-isomers of amino acids. A list of unnatural amino acid, contemplated herein is shown in Table 2.

TABLE 2
Codes for non-convention amino acids

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH2)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Natural product screening as well as the screening of chemical libraries is also a useful means of obtaining BMP4 chemical analogues as well as agonists or antagonists of BMP4. Natural product screening includes screening environments such as coral, sea and river beds, microorganisms, plants, rock formations or soil or rock from terrestrial or extraterrestrial (e.g., planetary environments or meteorites) for agents which function like BMP4 to differentiate stem cells into endodermal cells. These agents may also act as agonists to augment BMP4 activity. Antagonists are also contemplated in order to help control the differentiation process. The identification of functionally similar agents to BMP4 or agents which assist in the maintenance or expansion of mesodermal cells is described below.

The present invention extends to any mammalian stem cells such as from humans or other primates, livestock animals (e.g., sheep, pigs, cows, horses, goats, donkeys), laboratory test animals (eg. mice, rates, rabbits, hamsters, guinea pigs), companion animals (eg. dogs, cats) and captured wild animals. In so far as avian species have functionally equivalent cells to stem cells and mesodermal cells, the present invention extends to stem cells derived from avian species.

Reference to “mesodermal cells” includes any cell of mesodermal lineage such as mesendoderm, extraembryonic mesoderm and embryonic mesoderm as well as their partially or terminally differentiated progenitors.

The preferred cells from which differentiation is to be induced are EPL cells. However, other pluripotent or totipotent cells may also be used. Examples of these other cells include primitive ectoderm, primordial germ cells, embryonic germ cells, teratocarcinoma cells, ES cells, adult stem cells and pluripotent cells derived by nuclear reprogramming.

The properties of EPL cells, factors required for their maintenance and proliferation in vitro, and their ability to differentiate uniformly in vitro to form essentially homogeneous populations of partially differentiated and differentiated cell types are described in PCT/AU99/00265.

The pluripotent cell source may take the form of embryoid bodies (EB) derived from ES or EPL cells in vitro, or following cellular aggregation. Furthermore, pluripotent cells may be derived from EB cultured in MEDII (EBM).

BMP4 may be from any source such as commercial sources or from conditioned media or other natural sources.

The pluripotent EPL cells may be cultured according to the present invention under conditions suitable for their proliferation and maintenance in vitro. This includes the use of serum including fetal calf serum (FCS) and bovine serum or the medium may be serum-free. Other growth enhancing components such as insulin, transferrin and sodium selenite may be added to improve growth of the cell types preferred. As would be readily apparent to a person skilled in the art, the growth enhancing components will be dependent upon the cell types cultured, other growth factors present, attachment factors and amounts of serum present. Cytokines are particularly useful growth factors. Examples of cytokines include EPO, G-CSF, GM-CSF, FGF, Flt3, LIF, NIF, IGF, hedgehog, neuregulin, CTNF, NGF, TRH, EGF, TCF, PDGF, TGF-α, TGF-β, interleukins, SCF and nodal.

The EPL cells may be cultured for a time sufficient to establish the mesodermal cells in culture. By this is meant a time when the cells equilibrate in the culture medium. Preferably, the cells are cultured for approximately 2-6 days. The EPL cells are said to be “exposed” to BMP4. The exposure may occur in vitro, in vivo or ex vivo.

The cell culture medium may be any cell culture medium appropriate to sustain the EPL cells. In one embodiment, the culture medium is DMEM containing high glucose, 40 μg/ml gentamycin and 1 mM L-glutamine. The medium may contain up to 10% v/v FCS, but preferably the medium is serum free. Cultures are generally maintained at 37° C.

Separation of the cell culture medium from the cells may be achieved by any suitable technique, such as decanting the medium from the cells. Preferably the cell culture is clarified by centrifugation or filtration (eg. through a 0.22 μM filter) to remove excess cells and cellular debris. Other known means of separating the cells from the medium may be employed. A similar protocol is adopted when other stem cells are employed.

The culturing process may also include the addition of one or more growth factors such as those listed above.

The growth factor may also be selected to direct a specific mesodermal fate.

The present invention is further directed to mesodermal cells prepared by the process of culturing EPL cells or their committed progenitor cells in the presence of BMP4 for a time and under conditions sufficient for mesodermal cells to appear.

Generally, the mesodermal cells are defined by the expression of the mesodermal marker brachyury. The cells may also be defined by the absence of expression of certain genes such as SPARC and Collagen IV.

The identification of mesodermal cells as a differentiation product of EPL cells permits transcription and protein analysis to determine which genes are expressed and which are not expressed between different cell types or cells at different stages of development. This is referred to as a transcriptome or proteome profile.

Transcriptome and proteome profiles are useful in identifying genes or proteins or cell surface or sub-surface markers required to maintain a mesodermal cell in an undifferentiated state or to identify genes or proteins which are involved in differentiation, proliferation, expansion or renewal of mesodermal cells or stem cells (e.g., EPL cells).

Physiological changes associated with proteome analysis include screening for states of proliferation and/or differentiation. Immunological changes include changes in surface antigens which provides a profile of the developmental stage of the mesodermal or EPL or other stem cells. Examples of CD antigens, for example, which may be useful to monitor include CD3e, CD4, CD8a, CD11b, CD11c, CD15u, CD19, CD24, mCD301.1, CD31, CD34, CD41, CD45R, CD45RA, CD45RB, CD45RC, CD45RO, CD60a, CD60b, CD60c, CD75, CD75s, CD85, DC89, CD90.2, CD99R, CD117, CD110, CD111, CD112, CD117, CD133, CD135, CD156b, CD158, CD159a, CD160, CD162R, CD167a, CD168, CD69, CD123, CD170, CD171, CD172a, CD173, CD174, CD175, CD175s, CD176, CD177, CD178, CD179a, CD179b, CD180, CD183, CD184, CD195, CDw197, CD200, CD201, CD202b, CD203c, CD204, CD205, CD206, CD207, CD208, CD209, CDw210, CD212, CD213a1, CD213a2, CDw217, CD220, CD221, CD222, CD223, CD224, CD225, CD226, CD227, CD228, CD229, CD230, CD231, CD232, CD233, CD234, CD235a, CD235b, CD23ab, CD236, CD236R, CD238, CD239, CD240CE, CD240D, CD241, CD242, CD243, CD244, CD245, CD246 and CD247.

Assays measuring differentiation of stem cells or mesodermal cells include, for example, measuring cell-surface markers associated with stage-specific expression of a tissue, enzymatic activity, functional activity or morphological changes (Watt, FASEB 5: 281-284, 1991; Francis, Differentiation 57: 63-75, 1994; Raes, Adv. Anim. Cell Biol. Technol. Bioprocesses, 161-171, 1989). Assays measuring cell proliferation or differentiation include, for example, chemosensitivity to neutral red dye (Cavanaugh et al., Investigational New Drugs 8: 347-354. 1990), incorporation of radiolabeled nucleotides (Cook et al., Anal. Biochem. 179: 1-7, 1989), incorporation of 5-bromo-2′-deoxyuridine (BrdU) in the DNA of proliferating cells (Porstmann et al., J. Immunol. Methods 82: 169-179, 1985), and use of tetrazolium salts (Mosmann, J. Immunol. Methods 65: 55-63, 1983; Alley et al., Cancer Res. 48: 589-601, 1988; Marshall et al., Growth Reg. 5: 69-84, 1985; and Scudiero et al., Cancer Res. 48: 4827-4833, 1988) and by measuring proliferation using 3H-thymidine uptake (Crowley et al. J. Immunol. Meth. 133: 55-66, 1990).

Cell surface markers used for cell developmental stage determination may be labeled with a fluorescent compound. When the fluorescently labeled antibody or molecule with selective binding capacity is exposed to light of the proper wavelength, its presence can then be detected due to fluorescence. Among the most commonly used fluorescent labeling compounds are fluorescein isothiocyanate, rhodamine, isothiocyanate, phycoerythrin, phycocyanin, allophycocyanin, o-phthaldehyde and fluorescamine. The antibody or molecule with selective binding capacity can also be detectably labeled using fluorescence emitting metals such as ¹⁵²Eu or others of the lanthanide series. These metals can be attached to the antibody or molecule with selective binding capacity using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemilunescent-tagged antibody or molecule with selective binding capacity is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound can be used to label the antibody or molecule with selective binding capacity of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin. All such methods of labeling an antibody or a molecule with selective binding capacity are contemplated by the present invention.

Proteomics is a particularly useful way of studying changes from EPL or other stem cells to mesodermal cells as well as for monitoring the culture of mesodermal cells once obtained. Mesodermal cell cultures are conveniently assayed for maintenance of a level or stage of differentiation or non-differentiation.

Alternatively, agents can be screened for alterations to genetic material in EPL or other stem cells or mesodermal cells. For example, micro- or macro-array analysis and/or techniques such as differential hybridization, differential PCR and subtractive hybridization can be used to screen for transcripts present in proliferating and/or differentiating and/or renewing cells compared to resting cells. Once identified, the corresponding genes become specific targets for expression modulating agents to either facilitate or inhibit expression. Alternatively, EPL or other stem cells or mesodermal cells are exposed to potential agents and the changes in expression of genetic material monitored using, for example, differential expression protocols. The aim is to first find an agent which up- or down-regulates genetic material in, for example, an EPL or mesodermal cell and then determining whether this impacts on the developmental stage of the cell. Such agents are potential alternatives to BMP4.

Agents contemplated by the present invention include agonists and antagonists of specific target genes or gene products (e.g. receptors) such as antisense molecules, ribozymes, deoxyribozymes and minizymes and co-suppression molecules, RNAi, methylation promoting or inhibiting agents, peptides, polypeptides and proteins and chemical agents. Such agents may also be useful in de-differentiating or reprogramming a cell to an endodermal lineage pathway.

Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, carbohydrates, saccharides, fatty acids, steroids, purines, pyrimidines, derivatives, structural analogues or combinations thereof as well as components of the extracellular matrix. Biomolecules may also be isolated for traumatized or injured tissue or following immunological stimulation. Such biomolecules include inflammatory cytokines.

Small molecules are particularly preferred because such molecules are more readily absorbed after oral administration, have fewer potential antigenic determinants and/or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression) or affect the activity of a gene by inhibiting or enhancing the binding of accessory molecules.

Alternatively, libraries of natural compounds in the form of bacterial, fungal, plant and animal extracts, coral extracts and extracts from riverbeds, rocks and even extraterrestrial environments (e.g., from meteorites or samples from other planets) or may be used. Additionally, natural or synthetically produced libraries and compounds are readily modified through conventional chemical, physical and biochemical means, and may be used to produce combinatorial libraries. Known pharmacological agents, such as cytokines, may also be subjected to directed or random chemical modifications, such as acylation, alkylation, esterification, amidification, etc. to produce structural analogues.

Screening may also be directed to known pharmacologically active compounds and chemical analogues thereof.

Screening for modulatory agents according to the present invention can be achieved by any suitable method. For example, as indicated above, the method may include contacting an EPL or other stem cell or mesodermal cell and screening for the modulation of the level and/or functional activity of a protein encoded by a polynucleotide (this includes proteomics), or the modulation of the level of an expression product encoded by a polynucleotide, or the modulation of the activity or expression of a downstream cellular target of a protein or of an expression product or for a raft of physiological, biochemical, immunological or genetic changes including changes in surface antigen profiles (e.g. changes in CD antigen profile). All changes in expression in mesodermal cell genomes may be relative to ES cells, EPL cells or ectodermal cells or endodermal cells or other stem or mature cells. Detecting such modulation can be achieved by utilizing techniques including, but not restricted to, ELISA, cell-based ELISA, filter-binding ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

The present invention, therefore, provides assays for identifying small molecules or other compounds (i.e. modulatory agents) which are capable of inducing or inhibiting EPL or other stem cells or mesodermal cells proliferation and/or differentiation and/or self-renewal. The small molecules may also be useful for maintaining mesodermal cells at a particular stage or level of differentiation. The assays may be performed ex vivo using non-transformed cells lines, immortalized cell lines, recombinant cell lines or isolated cells. In addition, the assays may detect the presence of increased or decreased expression of genes or production of proteins on the basis of increased or decreased mRNA expression (using, for example, the nucleic acid probes), increased or decreased levels of protein products (using, for example, antigen-binding molecules) or increased or decreased levels of expression of a reporter gene (e.g., GFP, β-galactosidase or luciferase) operably linked to a target molecule-related gene regulatory region in a recombinant construct. An example of a target gene would be a receptor for an inflammatory cytokine.

Thus, for example, EPL or mesodermal cells which may be cultured or maintained in the presence of a particular target medium and a test compound added to the culture medium. After allowing a sufficient period of time (e.g. 1-200 hours) for the compound to induce or inhibit a physiological, biochemical, immunological or morphogical changes, any change from an established baseline may be detected using any of a range of macroscopic, microsopic techniques described above and well known in the art. In particularly preferred embodiments, the cells are stem cells or mature cells or cells developmentally in between. Using the nucleic acid probes and/or antigen-binding molecules for example, detection of changes in genetic expression or surface antigens can be readily detected.

In yet another embodiment, random peptide libraries consisting of all possible combinations of amino acids attached to a solid phase support may be used to identify peptides that are able to bind to a particular stem or mature cell surface antigen (which is indicative of a particular stage of development). The target antigen may be purified, recombinantly expressed or synthesized by any suitable technique. Such molecules may be conveniently prepared by a person skilled in the art using standard protocols as, for example, described in Sambrook, et al. (A Molecular Cloning—A Laboratory Manual, Cold Spring Harbour, N.Y., USA, 1989, in particular, Sections 16 and 17) and Ausubel et al., (“Current Protocols in Molecular Biology” John Wiley & Sons Inc, 1994-1998, in particular Chapters 10 and 16). Alternatively, a target antigen according to the invention may be synthesized using solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications and in Roberge et al. (Science 269: 202, 1995).

According to one particular embodiment, the present invention contemplates a method for screening for a change in developmental stage of an EPL or other stem cell or mesodermal cell, said method comprising exposing an in vitro or ex vivo culture or suspension of EPL or other stem cell or mesodermal cells to an agent having a potential to induce proliferation and/or differentiation and/or self-renewal wherein the level of proliferation and/or differentiation and/or self-renewal is determinable by a surface marker on said cells, contacting said cell surface with a ligand for a particular surface marker and then detecting the presence of binding to said surface marker wherein the pattern of surface markers determines whether an agent has induced proliferation and/or differentiation of said EPL or other stem cells.

In one embodiment, the surface marker is a CD antigen such as those listed above.

In another embodiment, the ligand is an antibody such as a monoclonal antibody.

In yet another embodiment, the EPL or other stem cell or mesodermal cells or cells developmentally in between after exposure to a potential proliferating- or differentiating- or self-renewal-stimulating agent is/are captured by immobilization to an anchored antibody to a solid support and then a range of antibodies labeled with separate reporter molecules or a range of anti-immunoglobulin antibodies each labeled with a reporter molecule are used to determine the existence of particular antigens which is indicative of the developmental stage of the cell.

In another embodiment, agents are first screened to the ability to alter expression of particular genetic sequences. Expressed sequence tags (ESTs) and cDNA libraries are particularly useful in analyzing the change in expression patterns of a cell.

Techniques based on cDNA substraction or differential display are quite useful for comparing gene expression differences between two cell types or between cells and different levels of development (Hendrick et al., Nature 308: 149, 1984; Liang and Pardee, Science 257: 967, 1992). The expressed sequence tag (EST) approach has been shown to be valuable tool for gene discovery (Adams et al., Science 252: 1656, 1992; Adams et al., Nature 355: 632, 1992; Okubo et al., Nature Genetics 2: 173, 1992) but like Northern blotting, RNase protection and reverse transcriptase-polymerase chain reaction (RT-PCR) analysis (Alwine et al., Proc. Natl. Acad. Sci. USA 74: 5350, 1977; Zinn et al., Cell 34:865, 1983; Verres et al., Science 237: 415, 1987) only evaluate a limited number of genes at a time. In addition, the EST approach preferably employs nucleotide sequences of 150 base pairs or longer for similarity searches and mapping.

Another valuable tool is serial analysis of gene expression (SAGE) [Velculescu et al., Science 270: 484-487, 1995; Velculescu et al., Cell 88: 243-251, 1997]. A modified protocol, called long SAGE may also be employed. SAGE is predicated in part on the use of short nucleotide sequences as a tag for transcript identification. Furthermore, the SAGE protocol generates concatemers of the short nucleotide sequences punctuated by known sequences. This permits the rapid and efficient sequencing of the nucleotide tags (Velculescu et al., 1995; supra; Velculescu et al., 1997, supra).

The present invention provides, therefore, a data set or library of nucleic acid molecules and/or nucleotide sequence information of these nucleic acid molecules from EPL or other stem cell or mesodermal cells generated from EPL cells following exposure to BMP4 or a homologue hereof or an agent as described above. The data set may comprise a single unique nucleic acid molecule or nucleotide sequence or the set may comprise hundreds or thousands of data units characteristic of a particular stage of development or indicative of proliferation and/or differentiation. Insofar as the data set comprises nucleic acid molecules, these may be in the form of a composition including being immoblized on a solid support such as an array of molecules on a chip or other planar or spherical surface.

The present invention provides, therefore, isolated nucleic acid molecules identified within a transcriptome profile of an EPL or other stem cell or an mesodermal cell. The transcriptome profile is conveniently generated by generating a population of cDNA molecules from mRNA isolated from EPL or other stem cell or mesodermal cells either exposed to or not exposed to a potential proliferation- and/or differentiation- and/or self-renewal-stimulating agent.

The present invention, therefore, contemplates a composition comprising a modulator of mesodermal cell generation from EPL or other stem cells or maintaining or expanding mesodermal cells said composition further comprising one or more pharmaceutically acceptable carriers and/or diluents. The composition may also affect cells developmentally between ES cells and mature cells.

The composition may also be in a multi-part pharmaceutical pack with instructions for use. In accordance with these instructions, two or more agents in the multi-part pack may be admixed together prior to use or given sequentially.

The compositions are proposed to be useful for a range or conditions, such as the repair, augmentation or regeneration of the haemopoietic lineage, muscle lineages, bone and connective tissue and organ tissue such as heart, liver, pancreas or kidney tissue as well as brain, epidermis, skin, breast, lung, head, thymus, eye, bone, epithelium, guts, biliary system, spleen and cells of the immune system. The compositions are generally used on cells ex vivo or in vitro which are then administered to a subject in need of the treatment.

The composition may also comprise genetic molecules such as a vector capable of transfecting target cells where the vector carries a nucleic acid molecule capable of encoding a modulator, when the modulator is a proteinaceous molecule. The vector may, for example, be a viral vector. In this regard, a range of gene therapies are contemplated by the present invention including isolating certain cells, genetically manipulating and returning the cell to the same subject or to a genetically related or similar subject.

Such information is also useful in reprogramming non-mesodermal cells into a mesodermal cell phenotype.

The isolated mesodermal cells or enriched endodermal cell population may, therefore, be maintained in vitro and optionally subjected to genetic manipulation. Alternatively, or in addition, the cells may be subjected to proliferation conditions and then used for tissue repair, regeneration and/or augmentation therapies.

Accordingly, another aspect of the present invention contemplates a method for tissue repair, regeneration and/or augmentation, said method comprising generating mesodermal cells by culturing stem cells in the presence of an effective amount of BMP4 or other agent as described herein for a time and under conditions sufficient to generate mesodermal cells optionally proliferating and/or further differentiating the mesodermal cells and then introducing the mesodermal cells into a subject requiring tissue repair, regeneration and/or augmentation.

More particularly, the present invention contemplates a method for tissue repair, regeneration and/or augmentation, said method comprising generating mesodermal cells by culturing EPL cells in the presence of an effective amount of BMP4 or other agent as described herein for a time and under conditions sufficient to generate mesodermal cells optionally proliferating and/or further differentiating the mesodermal cells and then introducing the mesodermal cells into a subject requiring tissue repair, regeneration and/or augmentation.

This aspect of the present invention contemplates “syngeneic”, “allogeneic” or “xenogeneic” transplantation with respect to the individuals within an animal species from which stem cells are isolated and the individuals who receive the cells. A “syngeneic” process means that the individual from which the stem cells are derived has the same MHC genotype as the recipient of derived mesodermal cells. An “allogeneic” process is where the stem cells are from a MHC-incompatible individual to the individual to which the derived mesodermal cells are to be introduced. A “xenogeneic” process is where the stem cells are from a different species to that to which the derived mesodermal cells are introduced. Preferably, the method of the present invention is conducted as a syngeneic process. To the extent that either an allogeneic or xenogeneic process is utilized, it should be understood that it may be necessary to modify the protocol such that any immunological responses, which may occur due to the mixing of foreign immuno-competent cells, are minimized. Because the invention also extends to autologous transplantations in which the cells transplanted are genetically identical to the recipients cells.

The present invention is further described by the following non-limiting Examples.

### EXAMPLE 1

Cell Culture

Feeder independent ES cell line D3 (Doetschman et al., Journal of Embryology and Experimental Morphology 87: 27-45, 1985) was used in this study. Routine maintenance of ES cells and the formation of EPL cells were performed as outlined in Smith et al., Dev. Biol. 151: 339-51, 1992 and Rathjen et al., Journal of Cell Science 112 (Pt 3): 601-12, 1999.

MEDII was produced as described in Rathjen et al., 1999, supra. Briefly, HepG2 cells (Knowles et al., Science 209: 497-499, 1980; ATCC HB-8065) were trypsinised to a single cell or near single cell suspension and seeded at 5×10⁴ cells/cm² in DMEM (Gibco BRL #12800) supplemented with 10% v/v fetal calf serum (FCS; Commonwealth Serum Laboratories) to give a ratio of 1.75×10⁵ cells/ml medium. Conditioned medium was collected after 4 days culture, sterilised by filtration through a 22 μm membrane and supplemented with 0.1 mM β-mercaptoethanol (β-ME) before use. MEDII was stored at 4° C. for 1-2 weeks. For these experiments MEDII was not frozen. HepG2 cells were replenished from frozen stocks every 2 months.

Embryoid Bodies formed in MEDII (EBM) were formed by aggregation of a single cell suspension of ES cells at a density of 1×10⁵ cells/ml in bacterial plates in 50% v/v MEDII media [50% v/v MEDII conditioned medium in DMEM (Gibco Dulbecco's Modified Eagle Medium without HEPES buffer) supplemented with 10% v/v FCS and 0.1 mM β-ME] at 37° C. in 10% v/v CO₂. EBMs were split 1 in 2 on the second day and medium was replaced on the third day. Using the EBM notation, the superscripted number refers to the number of days the aggregate has been in culture ie. EBM⁴ refers to EBM that have been cultured for 4 days.

### EXAMPLE 2

Differentiation Assays

EBM were aggregated and cultured for 3.5 days before being seeded individually into 2 ml wells of gelatin-treated (0.2% w/v gelatin in PBS for at least 30 minutes) tissue culture plastic in 50% MEDII. EBMs were allowed to adhere for 12 hours before the culture medium was removed and the aggregates were washed with PBS. The media was then replaced with a defined serum free medium [50% v/v DMEM, 50% v/v Hams F12 (Gibco BRL #11765) supplemented with 1×ITSS supplement (Boehringer Mannhiem)], with or without the addition of 10 ng/ml BMP4 (R&D Systems). Thus aggregates were exposed to BMP4 as EBM⁴. Aggregates were cultured until day 10 to 12 before being assessed for the presence of neural extensions or rhythmic contractions of cardiomyocytes, both identified by morphological critera. For each trial the percentage of aggregates showing neuron or cardiomyocyte formation was compared between those grown with and without 10 ng/ml BMP4. There were 7 separate trials, and the samples compared using a paired t-test for means.

### EXAMPLE 3

In Situ Hybridization Analysis

EBMs were aggregated and cultured for 3.5 days before being seeded as described above. The aggregates were grown for a further 2 days after the addition of serum free media with or without 10 ng/ml BMP4. They were then fixed with 4% w/v Paraformaldehyde in PBS for 15 minutes, before being dehydrated in 50% v/v ethanol in water for 15 minutes followed by 70% v/v ethanol in water. They were stored as seeded aggregates in 70% v/v ethanol in water at −20 ° C. until analysis. Prior to analysis aggregates were rehydrated to PBS with 0.1% v/v Tween through a wash in 50% v/v ethanol in water. Wholemount in situ hybridization analysis was performed as described in Lake et al., J. Cell. Sci 113 (Pt3): 555-566, 2000. Probes used were Oct4 (Rathjen et al., supra 1999) and brachyury (Lake et al., 2000 supra).

### EXAMPLE 4

BMP4 Induces the Formation of the Mesodermal Cell Population, Cardiomyocytes, From EBM4

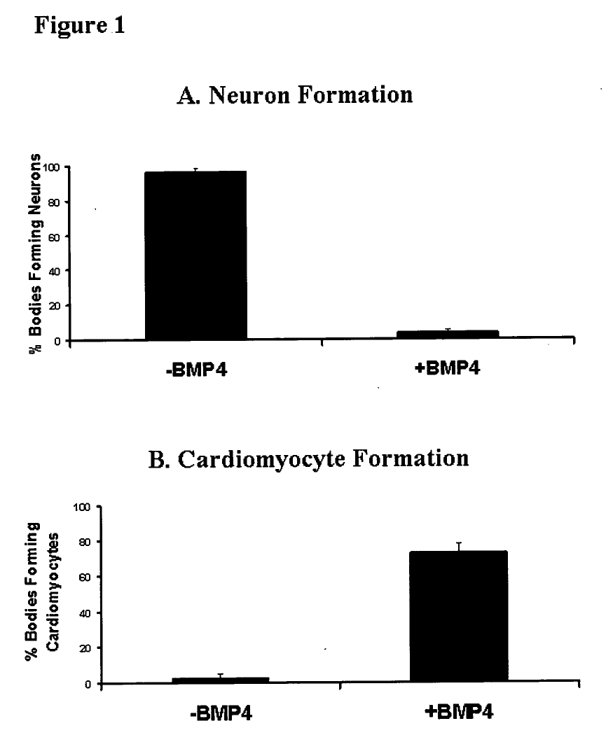

The formation of neurons was compared between EBM⁴s grown without BMP4 and with 10 ng/ml BMP4, as described above (FIG. 1A). When grown without BMP4, an average of 96.3% of aggregates formed neural extensions, compared to 3.6% of aggregates grown with BMP4 (P<0.01). The formation of cardiomyocytes was also compared (FIG. 1B). When grown without BMP4, an average of 2.4% of aggregates formed cardiomyocytes, compared to 72.9% of aggregates grown with BMP4 (P<0.01). These results indicate that the addition of 10 ng/ml BMP4 suppressed neuron formation and promoted cardiomyocytes, indicative of mesoderm formation.

### EXAMPLE 5

BMP4 Induces the Formation of Mesodermal Progenitors From EBM4 (EPL Cells)

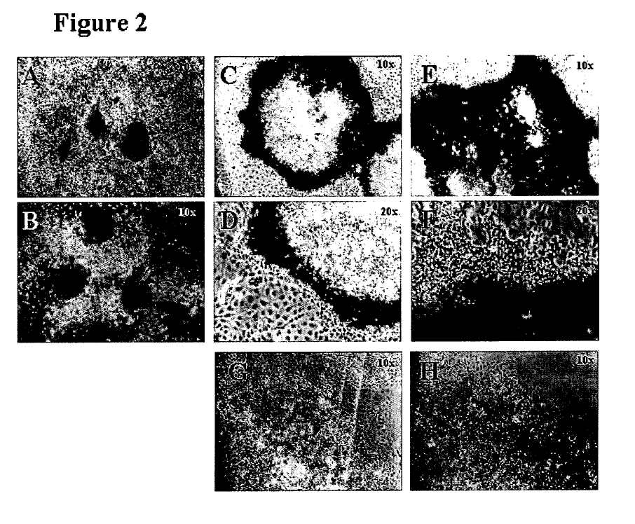

Gene expression analysis was performed via wholemount in situ hybridization on EBM⁴s that had been seeded and fixed as described above. Brachyury expression was used as a marker for nascent mesoderm (Herrmann, Development 113: 913-917, 1991), while Oct4 expression was used as a marker for pluripotent cells (Rosner et al., Nature 345: 686-692, 1990). Aggregates grown without BMP4 showed little expression of brachyury (FIG. 2A), and patchy areas of expression of Oct4 (FIG. 2B). In contrast, EBM⁴s grown in the presence of 10 ng/ml BMP4 developed a ring of cells around the aggregate that were strongly positive for brachyury FIGS. 2C,D). The cells of the aggregate, as well as those immediately surrounding it were strongly positive for Oct4 (Figure E,F). The ring of cells that were brachyury positive bordered the Oct4 positive population. Thus, when differentiated in the presence of BMP4, EBM⁴s produced a population of brachyury⁺ mesoderm precursors that surrounded a central population of Oct4⁺ pluripotent cells.

These results demonstrate that BMP4 can act on EBM⁴s to induce mesodermal precursors. EBMs have been shown to be EPL cells formed in suspension, equivalent to the primitive ectoderm population in the embryo. Thus, BMP4 can produce mesodermal precursors from EPL cells/primitive ectoderm.

### EXAMPLE 6

Treatment with BMP4 Results in the Phosphorylation of Smad 5

EBM Cell Lysis

EBMs were formed by aggregation and culture of ES cells, essentially as described above, for 3.5 days before being seeded onto gelatin-coated tissue culture plastic. After adhering for 12 hours the media was removed and replaced with serum free media (defined above) with or without the addition of 10 ng/mL BMP4 (R&D Systems). The cells were rinsed with PBS before the addition of serum free media to remove all traces of MEDII-containing media. After incubation at 37° C. for 15, 30, 45 and 120 minutes the media was removed, the cells were washed with PBS and then incubated in TEN buffer (40 mM Tris HCl pH 7.4, 1 mM EDTA, 150 mM NaCl) for 5-10 minutes at room temperature to lift the cells off the plastic. The cells were pelleted by centrifugation at 2000 rpm for 2 minutes, and washed several times with PBS. The cell pellets were snap-frozen using dry ice and ethanol and stored at −80° C.

Cell pellets were lysed with cell lysis buffer [20 mM HEPES, 0.42 M NaCl, 0.5% NP40, 25% Glycerol, 0.2 mM EDTA, 1.5 mM MgCl₂, MiniComplete™ protease inhibitor mix (Roche, 1 tablet per 10 mL), Na orthovanadate 1 mM, Na fluoride 15 mM] for 60 minutes at 4° C. with rotation. Cell debris was pelleted by centrifugation at 14,000 rpm for 15 minutes, and the supernatant removed to a clean tube. Total protein concentration was estimated by Bradford assay. Samples and standards were performed in duplicate. 10 μl of BSA standards (0-0.7 mg/ml) or samples (diluted 1:10 and 1:100) were mixed with 200 l of 1 in 4 diluted Bradford Reagent (BioRad) in a 96-well tray. Absorbance at 600 nm wavelength was measured in a Emax plate reader (Molecular Dynamics). Protein concentrations of samples were determined by calculation from the line of best fit of the standard curve, and equal amounts of protein were used for SDS-PAGE. Before SDS-PAGE the samples were mixed with an equal amount of 2×SDS loading buffer (125 mM Tris HCl pH 6.8, 4% (v/v) SDS, 20% (v/v) glycerol, 0.1% (w/v) bromophenol blue, 5% (v/v) β-mercaptoethanol) and boiled at 100° C. for 5 minutes.

SDS-PAGE

SDS-polyacrylamide gels [10% polyacrylamide (Protogel™, National Diagnostics), 3.75 M Tris HCl pH 8.8, 0.1% (w/v) SDS, 0.1% (w/v) APS, 0.1% (v/v) TEMED], were poured using 0.75 mm spacers and allowed to polymerise for approximately 20 minutes under a distilled water overlay. After polymerisation, the water was removed and a 4% stacker gel (4% polyacrylamide, 3.75 M Tris HCl pH 8.8, 0.1% (w/v) SDS, 0.1% (w/v) APS, 0.1% (v/v) TEMED) was applied. Ten well combs were inserted and the gel left to polymerise. Gels were electrophoresed using a PAGE minigel apparatus (BioRad) in SDS-PAGE buffer (25 mM Tris-Glycine, 0.1% (w/v) SDS) at 30-40 mAmps.

Western Blotting

Proteins were transferred from SDS-polyacrylamide gels to nitrocellulose (Protran, Schneider and Schell) in western transfer buffer (192 mM glycine, 25 mM Tris HCl pH 8.3, 0.1% (w/v) SDS, 20% (v/v) methanol), using a mini trans-blot electrophoretic transfer cell (BioRad). Filters were blocked by incubation in 5% (w/v) milk powder in PBT for 1 hour at room temperature. Primary antibody was added at an appropriate dilution, and the membrane was incubated overnight. Filters were washed using 3×20 minute washes in PBT before incubation with the appropriate HRP- or AP-conjugated secondary antibody. Secondary antibodies were diluted 1:2000 in either PBT (for HRP-conjugated antibodies) or TBST (for AP-conjugated antibodies) for 1 hour. Blots were developed after a further 3×20 minute washes in PBT or TBST. For HRP-conjugated secondary antibodies the blot was developed by bathing in enhanced chemiluminescence reagents for 5 minute (SuperSignal™ Substrates, Pearce), drained and exposed on autoradiographic film (Kodak/Fuji) for an appropriate time (1 second-5 minutes). For AP-conjugated secondary antibodies the blot was developed by bathing in chemifluorescent reagents for 5 minutes (ECF Substrate, Amersham Biosciences). The blots were then visualised by scanning with a Molecular Imager FX (BioRad) and analyzed using Quantity One (BioRad) software. Primary antibody dilutions used were:

| | |
|---|---|
| Goat anti-Smad 1 | 1:2000 in PBT with 1% (w/v) milk powder |
| (Santa Cruz) | |
| Goat anti-Smad 5 | 1:2000 in PBT |
| (Santa Cruz) | |
| Goat anti-Smad 8 | 1:2000 in PBT |
| (Santa Cruz) | |
| Goat anti-Actin | 1:2000 in PBT |
| (Santa Cruz) | |
| Rabbit anti-PhosphoSmad | 1:2000 in TBST with 5% BSA |
| (Cell Signaling | |
| Technology) | |

Immunoprecipitation

EBMs were aggregated and cultured as described above for 3.5 days before being seeded onto 10 cm gelatin-coated tissue culture dishes. After adhering for 12 hours the media was removed and replaced with serum free media (defined above) with or without the addition of 10 ng/mL BMP4 (R&D Systems). The cells were rinsed with PBS before the addition of serum free media to remove all traces of MEDII-containing media After incubation at 37° C. for 30 minutes the serum free media was removed and the cells were washed once with PBS. The cells were then lysed in the plate with 1 mL IP lysis buffer [50 mM Tris-HCl pH7.5, 150 mM NaCl, 10% (v/v) glycerol, 1% Triton X-100, 10 mM EDTA, MiniComplete™ protease inhibitor mix (Roche, 1 tablet per 10 mL), Na orthovanadate 1 mM, Na fluoride 15 mM] on ice for 30 minutes. Cell debris was pelleted by centrifugation at 14,000 rpm for 15 minutes at 4° C., and the supernatant removed to a clean tube. The lysate was precleared by incubating with 50 μL Protein A-Agarose (Roche) for 3 hours at 4° C., after which the agarose beads were pelleted by brief centrifugation and the supernatant transferred to a new tube. The cleared lysate was incubated with 5 μL of the appropriate primary antibody overnight at 4° C., after which 50 μL of Protein A-Agarose (Roche) was added and gently mixed for 3 hours, also at 4° C. After pelleting the agarose with brief centrifugation, the lysate was removed and the pellet washed twice for 20 minutes at 4° C. with chilled IP lysis buffer. After brief centrifugation, the final wash was removed, and the agarose resuspended with 50 μL of 2×SDS loading buffer (125 mM Tris HCl pH 6.8, 4% (v/v) SDS, 20% (v/v) glycerol, 0.1% (w/v) bromophenol blue, 5% (v/v) β-mercaptoethanol). This mix was boiled at 100° C. for 5 minutes before being subjected to SDS-PAGE and Western Blotting as described above.

BMP4 Signals via Smad 5 in EBM⁴ (EPL Cells)

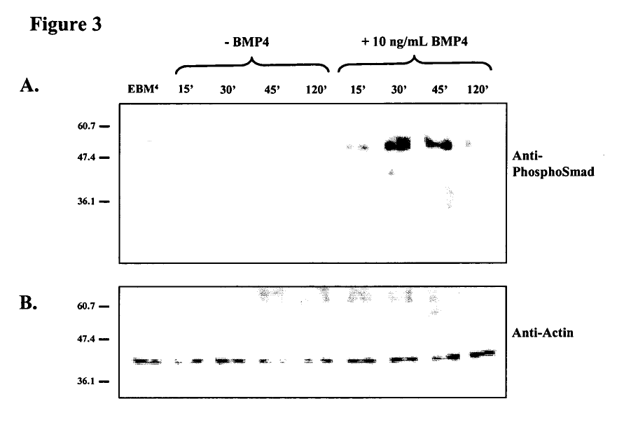

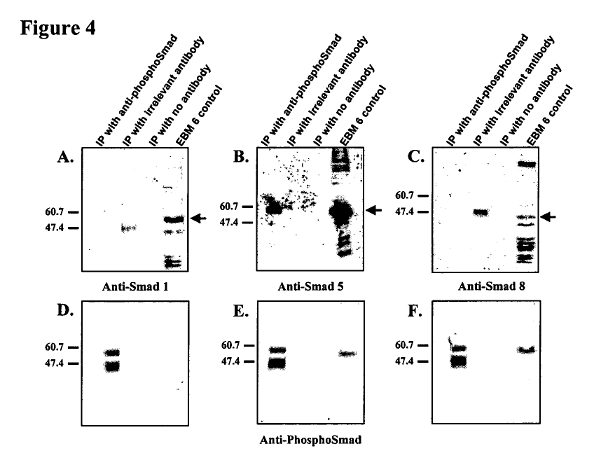

TGF-β family member ligands bind to a heteromeric receptor complex, activating an intracellular kinase domain which acts to phosphorylate Smad proteins. Phosphorylated Smad proteins are able to form a complex with Smad 4, which is then translocated to the nucleus to act as a transcriptional regulator. Smad 1, Smad 5 and Smad 8 phosphorylation is known to be restricted to BMP signalling (reviewed in Whitman, 1998). Protein extracts from EBM⁴s that had been grown with and without BMP4 were analyzed for the presence of phosphorylated Smads via Western Blot using an antibody which recognises only phosphorylated Smad 1, Smad 5 or Smad 8 (Cell Signaling Technology) (FIG. 3). Phosphorylated Smad protein was detected in aggregates that had been grown with BMP4, but not in those that had been grown without BMP4. To determine which of the BMP-specific Smads were being activated, the phosphorylated Smads were immunoprecipitated from cell lysates of EBM4s grown with BMP4 using the phosphoSmad antibody. The immunoprecipitated proteins were subjected to SDS-PAGE and analyzed for the presence of Smad 1, Smad 5 and Smad 8 by Western Blot (FIG. 4). Smad 5 was the only one detected. These results indicate that treatment of EBM⁴s (EPL cells) with BMP4 results in the phosphorylation of Smad 5.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

Bibliography

Adams et al., (1992) Science. 252: 1656.

Adams et al., (1992) Nature. 355: 632.

Alley et al., (1988) Cancer Res. 48: 589-601.

Alwine et al., (1977) Proc. Natl. Acad. Sci. USA 74: 5350.

Ausubel et al. “Current Protocols in Molecular Biology” John Wiley & Sons Inc. 1994-1998, in particular Chapters 10 and 16.

Cavanaugh et al., (1990) Investigational New Drugs. 8: 347-354.

Cook et al., (1989) Anal. Biochem. 179: 1-7.

Crowley et al., (1990) J. Immunol. Method. 133: 55-66.

Doetschman et al., (1985) J. of Embryology and Morph. 87, 27-45.

Francis, (1994) Differentiation 57: 63-75.

Hendrick et al., (1984) Nature. 308: 149.

Herrmann, B. G. (1991). Development. 113, 913-7.

Knowles et al., (1980) Science. 209, 497-9.

Lake et al., (2000) J. of Cell Science. 113 (Pt 3), 555-66.

Liang and Pardee, (1992) Science. 2577: 967.

Marshall et al., (1985) Growth Reg. 5: 69-84.

Mosmann, (1983) J. Immunol. Methods. 65: 55-63.

Okubo et al., (1992) Nature Genetics. 2: 173.

Portsmann et al., (1985) J. Immunol. Methods. 82: 169-179.

Raes, (1989) Adv. Anim. Cell Biol. Technol. Bioprocesses. 161-171.

Rathjen, J., et al., (1999). J. Cell Science. 112 (Pt 5), 601-12.

Roberge et al, (1995) Science. 269: 202.

Rosner et al., (1990) Nature. 345: 686-692.

Sambrook et al. “A Molecular Cloning—A Laboratory Manual”, Cold Spring Harbour, N.Y., USA 1989, in particular, Sections 16 and 17.

Scudiero et al, (1988) Cancer Res. 48: 4827-4833.

Smith et al., (1992). Dev. Biol. 151, 339-51.

Watt, (1991) FASEB 5: 281-284.

Whitman, M (1998) Genes and Dev. 12:2445-2462.

Velculescu et al., (1995) Science. 270: 484-487.

Velculescu et al., (1997) Cell. 88: 243-251.

Verres et al., (1987) Science. 237: 415.

Zinn et al., (1983) Cell. 34: 865.

What is claimed is:

1. A method for directing a population of cells to differentiate along a mesodermal cell lineage, said method comprising culturing said cells in the presence of bone morphogenetic protein 4 (BMP4) or a homologue, analogue or functional equivalent thereof for a time and under conditions sufficient for said cells to preferentially differentiate into mesodermal cells or cells of a mesodermal lineage.

2. The method of claim 1, wherein said cells are EPL cells.

3. The method of claim 1, wherein said cells are stem cells.

4. The method of claim 3, wherein said stem cells are selected from the group consisting of embryonic stem cells, somatic stem cells, germ stem cells, epidermal stem cells, adult neural stem cells, keratinocyte stem cells, melanocyte stem cells, adult renal stem cells, embryonic renal epithelial stem cells, embryonic endodermal stem cells, hepatocyte stem cells, mammary epithelial stem cells, bane marrow-derived stem cells, skeletal muscle stem cells, bone marrow mesenchymal stem cells, CD34⁺ haematopoietic stem cells and mesenchymal stem cells.

5. The method of claim 1, wherein said BMP4 is derived from a homologous species to said cells.

6. The method of claim 1, wherein said BMP4 is derived from a heterologous species to said cells.

7. The method of claims 1, wherein said cells are isolated from an animal selected form the group consisting of primates, livestock animals, laboratory test animals, companion animals and avian species.

8. The method of claim 7, wherein said cells are isolated from a mammal.

9. The method of claim 8, wherein said cells are isolated from a human.

10. A method for generating mesodermal cells from ES or EPL cells said method comprising:
(a) culturing ES cells or EPL cells in MEDII or its functional equivalent in order to generate embryoid bodies (EBM);
(b) maintaining said EBMs in culture for a time sufficient to allow aggregation of said EBMs;
(c) transferring said aggregated EBMs to gelatin-treated wells;
(d) allowing said aggregated EBMs to adhere to said gelatin-treated wells; and
(e) culturing said adhered EBMs in serum free medium comprising BMP4 for a time sufficient to allow said EBMs to generate mesodermal cells, and thereby generating mesodermal cells from ES cells or EPL cells.

11. The method of claim 10, wherein said BMP4 is derived from a species homologous to said cells.

12. The method of claim 10, wherein said BMP4 is derived from a species heterologous to said cells.

13. The method of claims 10, wherein said cells are isolated from an animal selected form the group consisting of primates, livestock animals, laboratory test animals, companion animals and avian species.

14. The method of claim 13, wherein said cells are isolated from a mammal.

15. The method of claim 14, wherein said cells are isolated from a human.

16. Mesodermal cells prepared by the process of culturing stem cells, or EPL cells or their committed progenitor cells in the presence of BMP4 for a time and under conditions sufficient for mesodermal cells to appear.

17. A method for screening for a change in a developmental stage of an EPL or other stem cell or mesodermal cell, said method comprising:
exposing an in vitro or ex vivo culture or suspension of EPL or other stem cell or mesodermal cells to an agent having a potential to induce proliferation and/or differentiation and/or self-renewal, wherein the level of proliferation and/or differentiation and/or self-renewal is determinable by a surface marker on said cells, contacting said cell surface with a ligand for said surface marker, and detecting the presence of binding to said surface marker, wherein the pattern of surface markers determines whether an agent has induced proliferation and/or differentiation of said EPL or other stem cell.

18. The method of claim 17; wherein said surface marker is specific for a mesodermal cell.

19. The method of claim 18, wherein said marker is brancyury.

20. The method of claim 17, wherein the stem cell is selected from the group consisting of: embryonic stem cells, somatic stem cells, germ stem cells, epidermal stem cells, adult neural stem cells, keratinocyte stem cells, melanocyte stem cells, adult renal stem cells, embryonic renal epithelial stem cells, embryonic endodermal stem cells, hepatocyte stem cells, mammary epithelial stem cells, bane marrow-derived stem cells, skeletal muscle stem cells, bone marrow mesenchymal stem cells, CD34⁺ haematopoietic stem cells and mesenchymal stem cells.

21. A method for determining a developmental stage of an EPL or other stem cell or mesodermal cell or cell developmentally in-between after exposure to a potential proliferating- or differentiating- or self-renewal-stimulating agent, said method comprising:
capturing said EPL or other stem cell or mesodermal cell or cell developmentally in-between by immobilization to an anchored antibody to a solid support, and
screening said immobilized cell with a range of antibodies labeled with separate reporter molecules or a range of anti-immunoglobulin antibodies each labeled with a reporter molecule used to determine existence of particular antigens said antigens being indicative of the developmental stage of the cell.

22. The method of claim 21, wherein said surface marker is specific for a mesodermal cell.

23. The method of claim 22, wherein said marker is brancyury.

24. The method of claim 21, wherein the stem cell is selected from the group consisting of: embryonic stem cells, somatic stem cells, germ stem cells, epidermal stem cells, adult neural stem cells, keratinocyte stem cells, melanocyte stem cells, adult renal stem cells, embryonic renal epithelial stem cells, embryonic endodermal stem cells, hepatocyte stem cells, mammary epithelial stem cells, bane marrow-derived stem cells, skeletal muscle stem cells, bone marrow mesenchymal stem cells, CD34⁺ haematopoietic stem cells and mesenchymal stem cells.

25. A method for tissue repair, regeneration and/or augmentation, said method comprising:
generating mesodermal cells by culturing EPL cells or stem cells in the presence of an effective amount of BMP4 or a functional equivalent thereof for a time and under conditions sufficient to generate mesodermal cells, and
introducing the mesodermal cells into a subject requiring tissue repair, regeneration and/or augmentation.

26. The method of claim 25, further comprising proliferating and/or further differentiating the mesodermal cells.

27. The method of claim 25, wherein said tissue is selected from the group consisting of cells of haemopoietic lineage, cells of muscle lineage, bone, connective tissue, organ tissue and cells of the immune system.

28. The method of claim 25, wherein said organ tissue is selected from heart, liver, pancreas, kidney, brain, epidermis, skin, breast, lung, head, thymus, eye, epithelium, gut, biliary system and spleen.

29. The method of claim 25, wherein the stem cell is selected from the group consisting of: embryonic stem cells, somatic stem cells, germ stem cells, epidermal stem cells, adult neural stem cells, keratinocyte stem cells, melanocyte stem cells, adult renal stem cells, embryonic renal epithelial stem cells, embryonic endodermal stem cells, hepatocyte stem cells, mammary epithelial stem cells, bane marrow-derived stem cells, skeletal muscle stem cells, bone marrow mesenchymal stem cells, CD34⁺ haematopoietic stem cells and mesenchymal stem cells.

30. The method of claim 25, wherein said BMP4 is derived from a species homologous to said stem cells or said EPL cells.

31. The method of claim 25, wherein said BMP4 is derived from a species heterologous to said stem cells or said EPL cells.

32. The method of claim 25, wherein said stem cell or said EPL cell is isolated from an animal selected from the group consisting of primates, livestock animals, laboratory test animals, companion animals and avian species.

33. The method of claim 32, wherein said stem cell or EPL cell is isolated from a mammal.

34. The method of claim 33, wherein said stem cell or EPL is isolated from a human.

35. A composition comprising a modulator of mesodermal cell generation from EPL or other stem cells or maintaining or expanding mesodermal cells said composition further comprising one or more pharmaceutically acceptable carriers and/or diluents.
